(19) United States
(12) Patent Application Publication
(10) Pub. No.: US 2023/0326021 A1
(43) Pub. Date: Oct. 12, 2023

(54) SYSTEM AND METHOD FOR NORMALIZING IMAGE DATA OBTAINED USING MULTISPECTRAL IMAGING

(71) Applicant: Leica Biosystems Imaging, Inc., Vista, CA (US)

(72) Inventor: Chad SALINAS, San Diego, CA (US)

(21) Appl. No.: 18/209,056

(22) Filed: Jun. 13, 2023

Related U.S. Application Data
(63) Continuation of application No. PCT/US2021/064361, filed on Dec. 20, 2021.
(60) Provisional application No. 63/131,243, filed on Dec. 28, 2020.

(51) Int. Cl. G06T 7/00; G16H 50/20

(52) U.S. Cl. CPC G06T 7/0012; G16H 50/20; G06T 2207/10036; G06T 2207/10024; G06T 2207/30024

(57) ABSTRACT

Systems and methods for normalizing image data obtained using multispectral imaging are disclosed. In one aspect, an image analysis apparatus includes an imaging device configured to perform a multispectral scan of a tissue sample to generate image data, a processor, and at least one computer-readable memory. The processor is configured to receive the image data from the imaging device and preprocess the image data to generate a normalization factor based on pixel values of the image data within at least one sub-region of the image data. The processor is also configured to provide the preprocessed image data and the normalization factor as inputs to a machine learning algorithm and determine, based on an output of the machine learning algorithm, whether the image data is indicative of a disease present in the tissue sample.

### CROSS-REFERENCE TO RELATED APPLICATION(S)

This application claims the benefit of U.S. Provisional Patent Application No. 63/131,243, filed Dec. 28, 2020, the disclosure of which is incorporated herein by reference.

### BACKGROUND

### Technical Field

The described technology relates to image data processing, and in particular, techniques for normalizing image data obtained using multispectral imaging of a tissue sample.

### Description of the Related Technology

Tissue samples can be analyzed under a microscope for various diagnostic purposes, including detecting cancer by identifying structural abnormalities in the tissue sample. A tissue sample can be imaged to produce image data using a microscope or other optical system. The image data can be analyzed using image processing techniques as a part of diagnosing whether the image data is indicative of a disease present in the tissue sample. Developments within the field of tissue sample diagnostics including the use of multispectral imaging are enabling more advanced diagnostic processes.

### SUMMARY

In one aspect, there is provided an image analysis apparatus, comprising: a memory coupled to an imaging device; and a hardware processor configured to: receive image data from the imaging device, the image data representative of a tissue sample, preprocess the image data to generate a normalization factor based on pixel values of the image data within at least one sub-region of the image data, provide the preprocessed image data and the normalization factor as inputs to a machine learning algorithm, and determine, based on an output of the machine learning algorithm, whether the image data is indicative of a disease present in the tissue sample.

The preprocessing the image data can comprise: subtracting at least one mathematical mean of the pixel values within the at least one sub-region of the image data from the pixel values.

The at least one mathematical mean can comprise a mathematical mean for each of three color channels of pixels values within the at least one sub-region.

The hardware processor is further configured to: initialize a plurality of weights applied to one or more inputs of activation nodes of the machine learning algorithm, wherein the plurality of weights facilitate confining the activation nodes within a defined Gaussian range.

The machine learning algorithm can be configured to interface with a plurality of layers of a neural network including an initial layer, one or more intermediate layers, and an output layer, and the hardware processor can be further configured to, for each of the one or more intermediate layers: take a random sample of input data to the one or more intermediate layers, calculate a mean and a variance of the random sample of the input data, and provide the mean and the variance as inputs to the one or more intermediate layers.

The one or more intermediate layers can comprise a convolution layer and one or more non-linear layers, and the hardware processor can be further configured to, for each of the one or more intermediate layers, scale input data to a corresponding intermediate layer by the normalization factor, the corresponding intermediate layer located after the convolution layer but before the one or more non-linear layers.

The machine learning algorithm can comprise a plurality of feature dimensions and a plurality of spatial locations, and the normalization factor can be applied individually for each of the feature dimensions and jointly for each of the spatial dimensions.

Each of the one or more intermediate layers can comprise one or more activation nodes, and the hardware processor can be further configured to, for each activation node, use the normalized feature dimensions and normalized spatial dimensions to scale and shift inputs to the activation node.

The hardware processor can be further configured to: identify wavelengths in the image data for which a threshold number of pixels have a gradient value in a same orientation vector.

The image analysis apparatus can further comprise: an imaging device configured to perform a multispectral scan of the tissue sample to generate the image data.

In another aspect, there is provided an image analysis apparatus, comprising: a memory coupled to an imaging device; and a hardware processor configured to: receive image data from the imaging device, the image data representative of a tissue sample, preprocess the image data to generate a normalization factor based on pixel values of the image data within at least one sub-region of the image data, and determine, based on the preprocessing image data and the normalization factor, whether the image data is indicative of a disease present in the tissue sample.

In yet another aspect, there is provided a method of determining whether a disease is present in a tissue sample, comprising: performing a multispectral scan of the tissue sample to generate image data; preprocessing the image data to generate a normalization factor based on pixel values of the image data within at least one sub-region of the image data; providing the preprocessed image data and the normalization factor as inputs to a machine learning algorithm; and determining, based on an output of the machine learning algorithm, whether the image data is indicative of a disease present in the tissue sample.

The preprocessing the image data can comprise: subtracting at least one mathematical mean of the pixel values within the at least one sub-region of the image data from the pixel values.

The at least one mathematical mean can comprise a mathematical mean for each of three color channels of pixels values within the at least one sub-region.

The method can further comprise: initializing a plurality of weights applied to one or more inputs of activation nodes of the machine learning algorithm, wherein the plurality of weights facilitate confining the activation nodes within a defined Gaussian range.

The machine learning algorithm can be configured to interface with a plurality of layers of a neural network including an initial layer, one or more intermediate layers, and an output layer, and the method can further comprise, for each of the one or more intermediate layers: taking a random sample of input data to the one or more intermediate layers; calculating a mean and a variance of the random sample of the input data; and providing the mean and the variance as inputs to the one or more intermediate layers.

The one or more intermediate layers can comprise a convolution layer and one or more non-linear layers, and the method can further comprise, for each of the one or more intermediate layers, scale input data to a corresponding intermediate layer by the normalization factor, the corresponding intermediate layer located after the convolution layer but before the one or more non-linear layers.

The machine learning algorithm can comprise a plurality of feature dimensions and a plurality of spatial locations, and the normalization factor can be applied individually for each of the feature dimensions and jointly for each of the spatial dimensions.

Each of the one or more intermediate layers can comprise one or more activation nodes, and the method can further comprise, for each activation node, using the normalized feature dimensions and normalized spatial dimensions to scale and shift inputs to the activation node.

The method can further comprise: identifying wavelengths in the image data for which a threshold number of pixels have a gradient value in a same orientation vector.

### BRIEF DESCRIPTION OF THE DRAWINGS

The features and advantages of the multi-stage stop devices, systems, and methods described herein will become more fully apparent from the following description and appended claims, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1 is illustrates an example environment in which a user and/or the multispectral imaging system may implement an image analysis system according to some embodiments.

FIG. 2 depicts an example workflow for generating image data from a tissue sample block according to some embodiments.

FIG. 3A illustrates an example prepared tissue block according to some embodiments.

FIG. 3B illustrates an example prepared tissue block and an example prepared tissue slice 300B according to some embodiments.

FIG. 4 shows an example imaging device, according to one embodiment.

FIG. 5 depicts a schematic diagram of an image analysis module, including multiple layers of a neural network in accordance with aspects of the present disclosure.

FIG. 6 is an example method for normalizing image data of a tissue sample obtained using multispectral imaging in accordance with aspects of this disclosure.

FIG. 7 is an example method for preprocessing the image data as a part of the method of FIG. 6 in accordance with aspects of this disclosure.

FIG. 8 is an example method for scaling input data provided to activation nodes of a machine learning algorithm as a part of the method of FIG. 6 in accordance with aspects of this disclosure.

FIG. 9 is an example computing system which can implement any one or more of the imaging device, image analysis system, and user computing device of the multispectral imaging system illustrated in FIG. 1.

### DETAILED DESCRIPTION

The features of the systems and methods for normalizing image data obtained using multispectral imaging will now be described in detail with reference to certain embodiments illustrated in the figures. The illustrated embodiments described herein are provided by way of illustration and are not meant to be limiting. Other embodiments can be utilized, and other changes can be made, without departing from the spirit or scope of the subject matter presented. It will be readily understood that the aspects and features of the present disclosure described below and illustrated in the figures can be arranged, substituted, combined, and designed in a wide variety of different configurations by a person of ordinary skill in the art, all of which are made part of this disclosure.

The diagnosis of tissue samples may involve a number of processing steps to prepare the tissue sample for viewing under a microscope. While the traditional diagnostics techniques may involve staining a tissue sample to provide additional visual contrast to the cellular structure of the sample when viewed under a microscope and manually diagnosing a disease by viewing the stained image through the microscope, multispectral imaging (also referred to as multispectral optical scanning) can be used to create image data which can be “virtually” stained using an image analysis system and provided to an image analysis system for processing. In some implementations, the image analysis system can include a machine learning algorithm trained to identifying and diagnose one or more diseases by identifying structures or features present in the image data that are consistent with training data used to train the machine learning algorithm.

Multispectral imaging may involve providing multispectral light to the tissue sample using a light source and detecting light emitted from the sample in response to the multispectral light using an imaging sensor. Under certain wavelengths/frequencies of the multispectral light, the tissue sample may exhibit autofluorescence which can be detected to generate image data that can be virtually stained. However, one or more of the processing steps for preparing the tissue sample for multispectral imaging may affect the image data obtained during a multispectral scan in a manner that can affect the image data in a consistent manner, which may result in autofluorescence patterns in the image data. For example, the fixation of the tissue sample using formalin may result in gradients in autofluorescence data that are not dependent on the structure of the tissue sample.

Aspects of this disclosure relate to systems and methods for prepressing the image data in order to compensate for one or more of the autofluorescence patterns in the image data generated due to tissue sample processing. Advantageously, aspects of this disclosure can increase the robustness of image processing of the image data to autofluorescence patterns due to pre-existing variation in time and spatial of the autofluorescence pattern due to tissue sample processing (e.g., due to formalin fixation across a tissue sample).

### System Overview

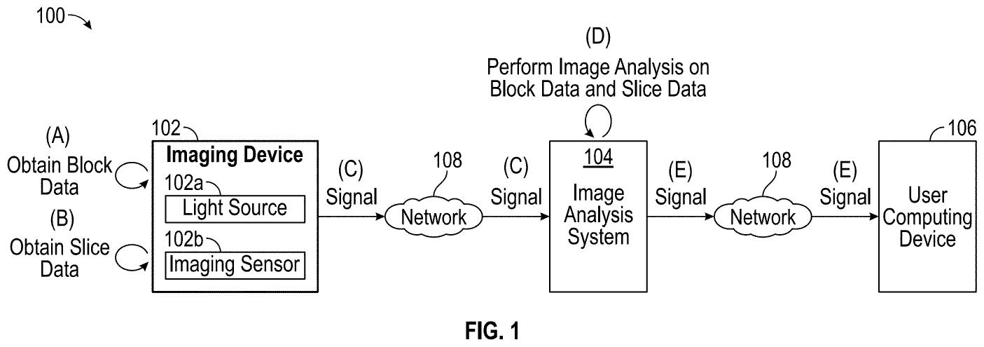

FIG. 1 is illustrates an example environment 100 (e.g., a multispectral imaging system) in which a user and/or the multispectral imaging system may implement an image analysis system 104 according to some embodiments. The image analysis system 104 may perform image analysis on received image data. The image analysis system 104 can normalize the image data obtained using multispectral imaging for input to a machine learning algorithm. Based on the normalized image data, the machine learning algorithm can diagnose whether the image data is indicative of a disease present in the tissue sample.

The image analysis system 104 may perform the image analysis using an image analysis module (not shown in FIG. 1). The image analysis system 104 may receive the image data from an imaging device 102 and transmit the recommendation to a user computing device 106 for processing. Although some examples herein refer to a specific type of device as being the imaging device 102, the image analysis system 104, or the user computing device 106, the examples are illustrative only and are not intended to be limiting, required, or exhaustive. The image analysis system 104 may be any type of computing device (e.g., a server, a node, a router, a network host, etc.). Further, the imaging device 102 may be any type of imaging device (e.g., a camera, a scanner, a mobile device, a laptop, etc.). In some embodiments, the imaging device 102 may include a plurality of imaging devices. Further, the user computing device 106 may be any type of computing device (e.g., a mobile device, a laptop, etc.).

In some implementations, the imaging device 102 includes a light source 102a configured to emit multispectral light onto the tissue sample(s) and the image sensor 102b configured to detect multispectral light emitted from the tissue sample. The multispectral imaging using the light source 102a can involve providing light to the tissue sample carried by a carrier within a range of frequencies. That is, the light source 102a may be configured to generate light across a spectrum of frequencies to provide multispectral imaging.

In certain embodiments, the tissue sample may reflect light received from the light source 102a, which can then be detected at the image sensor 102b. In these implementations, the light source 102a and the image sensor 102b may be located on substantially the same side of the tissue sample. In other implementations, the light source 102a and the image sensor 102b may be located on opposing sides of the tissue sample. The image sensor 102b may be further configured to generate image data based on the multispectral light detected at the image sensor 102b. In certain implementations, the image sensor 102b may include a high-resolution sensor configured to generate a high-resolution image of the tissue sample. The high-resolution image may be generated based on excitation of the tissue sample in response to laser light emitted onto the sample at different frequencies (e.g., a frequency spectrum).

The imaging device 102 may capture and/or generate image data for analysis. The imaging device 102 may include one or more of a lens, an image sensor, a processor, or memory. The imaging device 102 may receive a user interaction. The user interaction may be a request to capture image data. Based on the user interaction, the imaging device 102 may capture image data. In some embodiments, the imaging device 102 may capture image data periodically (e.g., every 10, 20, or 30 minutes). In other embodiments, the imaging device 102 may determine that an item has been placed in view of the imaging device 102 (e.g., a histological sample has been placed on a table and/or platform associated with the imaging device 102) and, based on this determination, capture image data corresponding to the item. The imaging device 102 may further receive image data from additional imaging devices. For example, the imaging device 102 may be a node that routes image data from other imaging devices to the image analysis system 104. In some embodiments, the imaging device 102 may be located within the image analysis system 104. For example, the imaging device 102 may be a component of the image analysis system 104. Further, the image analysis system 104 may perform an imaging function. In other embodiments, the imaging device 102 and the image analysis system 104 may be connected (e.g., wirelessly or wired connection). For example, the imaging device 102 and the image analysis system 104 may communicate over a network 108. Further, the imaging device 102 and the image analysis system 104 may communicate over a wired connection. In one embodiment, the image analysis system 104 may include a docking station that enables the imaging device 102 to dock with the image analysis system 104. An electrical contact of the image analysis system 104 may connect with an electrical contact of the imaging device 102. The image analysis system 104 may be configured to determine when the imaging device 102 has been connected with the image analysis system 104 based at least in part on the electrical contacts of the image analysis system 104. In some embodiments, the image analysis system 104 may use one or more other sensors (e.g., a proximity sensor) to determine that an imaging device 102 has been connected to the image analysis system 104. In some embodiments, the image analysis system 104 may be connected to (via a wired or a wireless connection) a plurality of imaging devices.

The image analysis system 104 may include various components for providing the features described herein. In some embodiments, the image analysis system 104 may include one or more image analysis modules to perform the image analysis of the image data received from the imaging device 102. The image analysis modules may perform one or more imaging algorithms using the image data.

The image analysis system 104 may be connected to the user computing device 106. The image analysis system 104 may be connected (via a wireless or wired connection) to the user computing device 106 to provide a recommendation for a set of image data. The image analysis system 104 may transmit the recommendation to the user computing device 106 via the network 108. In some embodiments, the image analysis system 104 and the user computing device 106 may be configured for connection such that the user computing device 106 can engage and disengage with image analysis system 104 in order to receive the recommendation. For example, the user computing device 106 may engage with the image analysis system 104 upon determining that the image analysis system 104 has generated a recommendation for the user computing device 106. Further, a particular user computing device 106 may connect to the image analysis system 104 based on the image analysis system 104 performing image analysis on image data that corresponds to the particular user computing device 106. For example, a user may be associated with a plurality of histological samples. Upon determining, that a particular histological sample is associated with a particular user and a corresponding user computing device 106, the image analysis system 104 can transmit a recommendation for the histological sample to the particular user computing device 106. In some embodiments, the user computing device 106 may dock with the image analysis system 104 in order to receive the recommendation.

In some implementations, the imaging device 102, the image analysis system 104, and/or the user computing device 106 may be in wireless communication. For example, the imaging device 102, the image analysis system 104, and/or the user computing device 106 may communicate over a network 108. The network 108 may include any viable communication technology, such as wired and/or wireless modalities and/or technologies. The network may include any combination of Personal Area Networks (“PANs”), Local Area Networks (“LANs”), Campus Area Networks (“CANs”), Metropolitan Area Networks (“MANs”), extranets, intranets, the Internet, short-range wireless communication networks (e.g., ZigBee, Bluetooth, etc.), Wide Area Networks (“WANs”)— both centralized and/or distributed—and/or any combination, permutation, and/or aggregation thereof. The network 108 may include, and/or may or may not have access to and/or from, the internet. The imaging device 102 and the image analysis system 104 may communicate image data. For example, the imaging device 102 may communicate image data associated with a histological sample to the image analysis system 104 via the network 108 for analysis. The image analysis system 104 and the user computing device 106 may communicate a recommendation corresponding to the image data. For example, the image analysis system 104 may communicate a diagnosis regarding whether the image data is indicative of a disease present in the tissue sample based on the results of a machine learning algorithm. In some embodiments, the imaging device 102 and the image analysis system 104 may communicate via a first network and the image analysis system 104 and the user computing device 106 may communicate via a second network. In other embodiments, the imaging device 102, the image analysis system 104, and the user computing device 106 may communicate over the same network.

With reference to an illustrative embodiment, at [A], the imaging device 102 can obtain block data. In order to obtain the block data, the imaging device 102 can image (e.g., scan, capture, record, etc.) a tissue block. The tissue block may be a histological sample. For example, the tissue block may be a block of biological tissue that has been removed and prepared for analysis. As will be discussed in further below, in order to prepare the tissue block for analysis, various histological techniques may be performed on the tissue block. The imaging device 102 can capture an image of the tissue block and store corresponding block data in the imaging device 102. The imaging device 102 may obtain the block data based on a user interaction. For example, a user may provide an input through a user interface (e.g., a graphical user interface (“GUI”)) and request that the imaging device 102 image the tissue block. Further, the user can interact with imaging device 102 to cause the imaging device 102 to image the tissue block. For example, the user can toggle a switch of the imaging device 102, push a button of the imaging device 102, provide a voice command to the imaging device 102, or otherwise interact with the imaging device 102 to cause the imaging device 102 to image the tissue block. In some embodiments, the imaging device 102 may image the tissue block based on detecting, by the imaging device 102, that a tissue block has been placed in a viewport of the imaging device 102. For example, the imaging device 102 may determine that a tissue block has been placed on a viewport of the imaging device 102 and, based on this determination, image the tissue block.

At [B], the imaging device 102 can obtain slice data. In some embodiments, the imaging device 102 can obtain the slice data and the block data. In other embodiments, a first imaging device can obtain the slice and a second imaging device can obtain the block data. In order to obtain the slice data, the imaging device 102 can image (e.g., scan, capture, record, etc.) a slice of the tissue block. The slice of the tissue block may be a slice of the histological sample. For example, the tissue block may be sliced (e.g., sectioned) in order to generate one or more slices of the tissue block. In some embodiments, a portion of the tissue block may be sliced to generate a slice of the tissue block such that a first portion of the tissue block corresponds to the tissue block imaged to obtain the block data and a second portion of the tissue block corresponds to the slice of the tissue block imaged to obtain the slice data. As will be discussed in further detail below, various histological techniques may be performed on the tissue block in order to generate the slice of the tissue block. The imaging device 102 can capture an image of the slice and store corresponding slice data in the imaging device 102. The imaging device 102 may obtain the slice data based on a user interaction. For example, a user may provide an input through a user interface and request that the imaging device 102 image the slice. Further, the user can interact with imaging device 102 to cause the imaging device 102 to image the slice. In some embodiments, the imaging device 102 may image the tissue block based on detecting, by the imaging device 102, that the tissue block has been sliced or that a slice has been placed in a viewport of the imaging device 102.

At [C], the imaging device 102 can transmit a signal to the image analysis system 104 representing the captured image data (e.g., the block data and the slice data). The imaging device 102 can send the captured image data as an electronic signal to the image analysis system 104 via the network 108. The signal may include and/or correspond to a pixel representation of the block data and/or the slice data. It will be understood that the signal can include and/or correspond to more, less, or different image data. For example, the signal may correspond to multiple slices of a tissue block and may represent a first slice data and a second slice data. Further, the signal may enable the image analysis system 104 to reconstruct the block data and/or the slice data. In some embodiments, the imaging device 102 can transmit a first signal corresponding to the block data and a second signal corresponding to the slice data. In other embodiments, a first imaging device can transmit a signal corresponding to the block data and a second imaging device can transmit a signal corresponding to the slice data.

At [D], the image analysis system 104 can perform image analysis on the block data and the slice data provided by the imaging device 102. In order to perform the image analysis, the image analysis system 104 may utilize one or more image analysis modules that can perform one or more image processing functions. For example, the image analysis module may include an imaging algorithm, a machine learning model, a convolutional neural network, or any other modules for performing the image processing functions. Based on performing the image processing functions, the image analysis module can determine a likelihood that the block data and the slice data correspond to the same tissue block. For example, an image processing functions may include an edge analysis of the block data and the slice data and based on the edge analysis, determine whether the block data and the slice data correspond to the same tissue block. The image analysis system 104 can obtain a confidence threshold from the user computing device 106, the imaging device 102, or any other device. In some embodiments, the image analysis system 104 can determine the confidence threshold based on a response by the user computing device 106 to a particular recommendation. Further, the confidence threshold may be specific to a user, a group of users, a type of tissue block, a location of the tissue block, or any other factor. The image analysis system 104 can compare the determined confidence threshold with the image analysis performed by the image analysis module. Based on this comparison, the image analysis system 104 can generate a recommendation indicating a recommended action for the user computing device 106 based on the likelihood that the block data and the slice data correspond to the same tissue block. In other embodiments, the image analysis system 104 can provide a diagnosis regarding whether the image data is indicative of a disease present in the tissue sample, for example, based on the results of a machine learning algorithm.

At [E], the image analysis system 104 can transmit a signal to the user computing device 106. The image analysis system 104 can send the signal as an electrical signal to the user computing device 106 via the network 108. The signal may include and/or correspond to a representation of the diagnosis. Based on receiving the signal, the user computing device 106 can determine the diagnosis. In some embodiments, the image analysis system 104 may transmit a series of recommendations corresponding to a group of tissues blocks and/or a group of slices. The image analysis system 104 can include, in the recommendation, a recommended action of a user. For example, the recommendation may include a recommendation for the user to review the tissue block and the slice. Further, the recommendation may include a recommendation that the user does not need to review the tissue block and the slice.

### Imaging Prepared Blocks and Prepared Slices

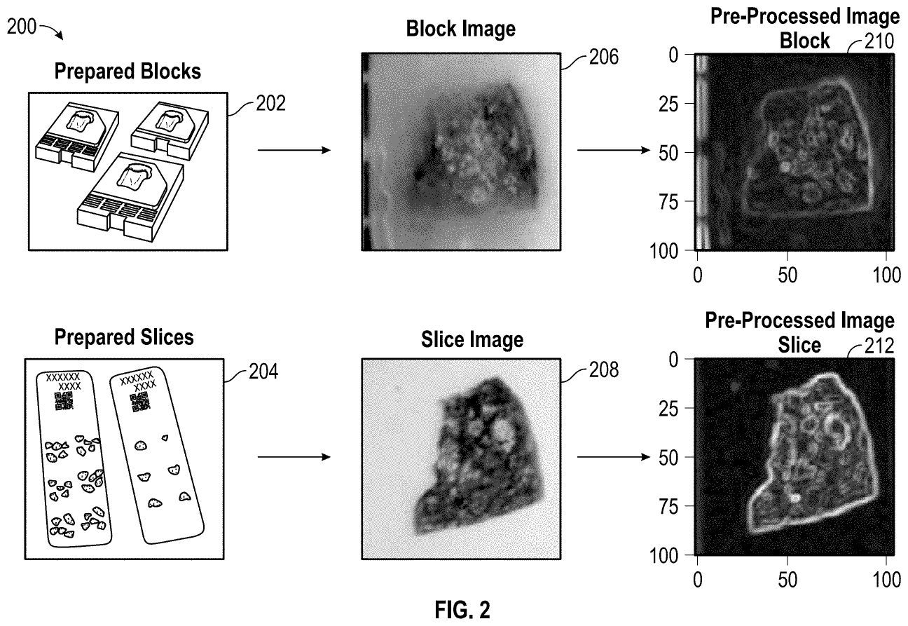

FIG. 2 depicts an example workflow 200 for generating image data from a tissue sample block according to some embodiments. The example workflow 200 illustrates a process for generating prepared blocks and prepared slices from a tissue block and generating pre-processed images based on the prepared blocks and the prepared slices. The example workflow 200 may be implemented by one or more computing devices. For example, the example workflow 200 may be implemented by a microtome, a coverslipper, a stainer, and an imaging device. Each computing device may perform a portion of the example workflow. For example, the microtome may cut the tissue block in order to generate one or more slices of the tissue block. The coverslipper may create a first slide for the tissue block and/or a second slide for a slice of the tissue block, the stainer may stain each slide, and the imaging device may image each slide.

A tissue block can be obtained from a patient (e.g., a human, an animal, etc.). The tissue block may correspond to a section of tissue from the patient. The tissue block may be surgically removed from the patient for further analysis. For example, the tissue block may be removed in order to determine if the tissue block has certain characteristics (e.g., if the tissue block is cancerous). In order to generate the prepared blocks 202, the tissue block may be prepared using a particular preparation process by a tissue preparer. For example, the tissue block may be preserved and subsequently embedded in a paraffin wax block. Further, the tissue block may be embedded (in a frozen state or a fresh state) in a block. The tissue block may also be embedded using an optimal cutting temperature (“OCT”) compound. The preparation process may include one or more of a paraffin embedding, an OCT-embedding, or any other embedding of the tissue block. In the example of FIG. 2, the tissue block is embedded using paraffin embedding. Further, the tissue block is embedded within a paraffin wax block and mounted on a microscopic slide in order to formulate the prepared block.

The microtome can obtain a slice of the tissue block in order to generate the prepared slices 204. The microtome can use one or more blades to slice the tissue block and generate a slice (e.g., a section) of the tissue block. The microtome can further slice the tissue block to generate a slice with a preferred level of thickness. For example, the slice of the tissue block may be 1 millimeter. The microtome can provide the slice of the tissue block to a coverslipper. The coverslipper can encase the slice of the tissue block in a slide to generate the prepared slices 204. The prepared slices 204 may include the slice mounted in a certain position. Further, in generating the prepared slices 204, a stainer may also stain the slice of the tissue block using any staining protocol. Further, the stainer may stain the slice of the tissue block in order to highlight certain portions of the prepared slices 204 (e.g., an area of interest). In some embodiments, a computing device may include both the coverslipper and the stainer and the slide may be stained as part of the process of generating the slide.

The prepared blocks 202 and the prepared slices 204 may be provided to an imaging device for imaging. In some embodiments, the prepared blocks 202 and the prepared slices 204 may be provided to the same imaging device. In other embodiments, the prepared blocks 202 and the prepared slices 204 are provided to different imaging devices. The imaging device can perform one or more imaging operations on the prepared blocks 202 and the prepared slices 204. In some embodiments, a computing device may include one or more of the tissue preparer, the microtome, the coverslipper, the stainer, and/or the imaging device.

The imaging device can capture an image of the prepared block 202 in order to generate the block image 206. The block image 206 may be a representation of the prepared block 202. For example, the block image 206 may be a representation of the prepared block 202 from one direction (e.g., from above). The representation of the prepared block 202 may correspond to the same direction as the prepared slices 204 and/or the slice of the tissue block. For example, if the tissue block is sliced in a cross-sectional manner in order to generate the slice of the tissue block, the block image 206 may correspond to the same cross-sectional view. In order to generate the block image 206, the prepared block 202 may be placed in a cradle of the imaging device and imaged by the imaging device. Further, the block image 206 may include certain characteristics. For example, the block image 206 may be a color image with a particular resolution level, clarity level, zoom level, or any other image characteristics.

The imaging device can capture an image of the prepared slices 204 in order to generate the slice image 208. The imaging device can capture an image of a particular slice of the prepared slices 204. For example, a slide may include any number of prepared slices and the imaging device may capture an image of a particular slice of the prepared slices. The slice image 208 may be a representation of the prepared slices 204. The slice image 208 may correspond to a view of the slice according to how the slice of the tissue block was generated. For example, if the slice of the tissue block was generated via a cross-sectional cut of the tissue block, the slice image 208 may correspond to the same cross-sectional view. In order to generate the slice image 208, the slide containing the prepared slices 204 may be placed in a cradle of the imaging device (e.g., in a viewer of a microscope) and imaged by the imaging device. Further, the slice image 208 may include certain characteristics. For example, the slice image 208 may be a color image with a particular resolution level, clarity level, zoom level, or any other image characteristics.

The imaging device can process the block image 206 in order to generate a pre-processed image 210 and the slice image 208 in order to generate the pre-processed image 212. The imaging device can perform one or more image operations on the block image 206 and the slice image 208 in order to generate the pre-processed image 210 and the pre-processed image 212. The one or more image operations may include isolating (e.g., focusing on) various features of the pre-processed image 210 and the pre-processed imaged 212. For example, the one or more image operations may include isolating the edges of a slice or a tissue block, isolating areas of interest within a slice or a tissue block, or otherwise modifying (e.g., transforming) the block image 206 and/or the slice image 208. In some embodiments, the imaging device can perform the one or more image operations on one of the block image 206 or the slice image 208. For example, the imaging may perform the one or more image operations on the block image 206. In other embodiments, the imaging device can perform first image operations on the block image 206 and second image operations on the slice image 208. The imaging device may provide the pre-processed image 210 and the pre-processed image 212 to the image analysis system to determine a likelihood that the pre-processed image 210 and the pre-processed image 212 correspond to the same tissue block.

### Slicing a Tissue Block

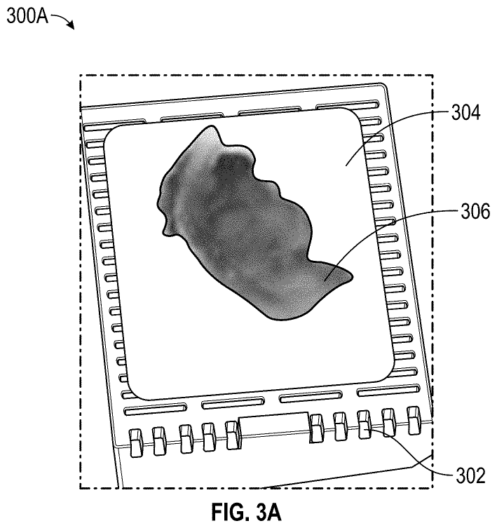

FIG. 3A illustrates an example prepared tissue block 300A according to some embodiments. The prepared tissue block 300A may include a tissue block 306 that is preserved (e.g., chemically preserved, fixed, supported) in a particular manner. In order to generate the prepared tissue block 300A, the tissue block 306 can be placed in a fixing agent (e.g., a liquid fixing agent). For example, the tissue block 306 can be placed in a fixative such as formaldehyde solution. The fixing agent can penetrate the tissue block 306 and preserve the tissue block 306. The tissue block 306 can subsequently be isolated in order to enable further preservation of the tissue block 306. Further, the tissue block 306 can be immersed in one or more solutions (e.g., ethanol solutions) in order to replace water within the tissue block 306 with the one or more solutions. The tissue block 306 can be immersed in one or more intermediate solutions. Further, the tissue block 306 can be immersed in a final solution (e.g., a histological wax). For example, the histological wax may be a purified paraffin wax. After being immersed in a final solution, the tissue block 306 may be formed into a prepared tissue block 300A. For example, the tissue block 306 may be placed into a mould filled with the histological wax. By placing the tissue block in the mould, the tissue block 306 may be moulded (e.g., encased) in the final solution 304. In order to generate the prepared tissue block 300A, the tissue block 306 in the final solution 304 may be placed on a platform 302. Therefore, the prepared tissue block 300A may be generated. It will be understood that the prepared tissue block 300A may be prepared according to any tissue preparation methods.

FIG. 3B illustrates an example prepared tissue block 300A and an example prepared tissue slice 300B according to some embodiments. The prepared tissue block 300A may include the tissue block 306 encased in a final solution 304 and placed on a platform 302. In order to generate the prepared tissue slice 300B, the prepared tissue block 300A may be sliced by a microtome. The microtome may include one or more blades to slice the prepared tissue block 300A. The microtome may take a cross-sectional slice 310 of the prepared tissue block 300A using the one or more blades. The cross-sectional slice 310 of the prepared tissue block 300A may include a slice 310 (e.g., a section) of the tissue block 306 encased in a slice of the final solution 304. In order to preserve the slice 310 of the tissue block 306, the slice 310 of the tissue block 306 may be modified (e.g., washed) to remove the final solution 304 from the slice 310 of the tissue block 306. For example, the final solution 304 may be rinsed and/or isolated from the slice 310 of the tissue block 306. Further, the slice 310 of the tissue block 306 may be stained by a stainer. In some embodiments, the slice 310 of the tissue block 306 may not be stained. The slice 310 of the tissue block 306 may subsequently be encased in a slide 308 by a coverslipper to generate the prepared tissue slice 300B. The prepared tissue slice 300B may include an identifier 312 (also referred to as tissue slide identifying information) identifying the tissue block 306 that corresponds to the prepared tissue slice 300B. Not shown in FIG. 3B, the prepared tissue block 300A may also include an identifier that identifies the tissue block 306 that corresponds to the prepared tissue block 300A. As the prepared tissue block 300A and the prepared tissue slice 300B correspond to the same tissue block 306, the identifier of the prepared tissue block 300A and the identifier 312 of the prepared tissue slice 300B may identify the same tissue block 306.

### Imaging Devices

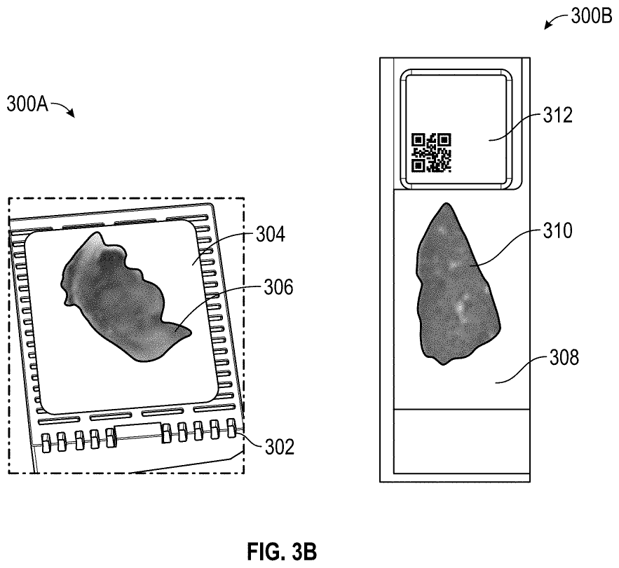

FIG. 4 shows an example imaging device 400, according to one embodiment. The imaging device 400 can include an imaging apparatus 402 (e.g., a lens and an image sensor) and a platform 404. The imaging device 400 can receive a prepared tissue block and/or a prepared tissue slice via the platform 404. Further, the imaging device can use the imaging apparatus 402 to capture image data corresponding to the prepared block and/or the prepared slice. The imaging device 400 can be one or more of a camera, a scanner, a medical imaging device, etc. Further, the imaging device 400 can use imaging technologies such as X-ray radiography, magnetic resonance imaging, ultrasound, endoscopy, elastography, tactile imaging, thermography, medical photography, nuclear medicine functional imaging, positron emission tomography, single-photon emission computed tomography, etc. For example, the imaging device can be a magnetic resonance imaging (“MRI”) scanner, a positron emission tomography (“PET”) scanner, an ultrasound imaging device, an x-ray imaging device, a computerized tomography (“CT”) scanner,

The imaging device 400 may receive one or more of the prepared tissue block and/or the prepared tissue slice and capture corresponding image data. In some embodiments, the imaging device 400 may capture image data corresponding to a plurality of prepared tissue slices and/or a plurality of prepared tissue blocks. The imaging device 400 may further capture, through the lens of the imaging apparatus 402, using the image sensor of the imaging apparatus 402, a representation of a prepared tissue slice and/or a prepared tissue block as placed on the platform. Therefore, the imaging device 400 can capture image data in order for the image analysis system to compare the image data to determine if the image data corresponds to the same tissue block.

### Machine Learning Algorithms

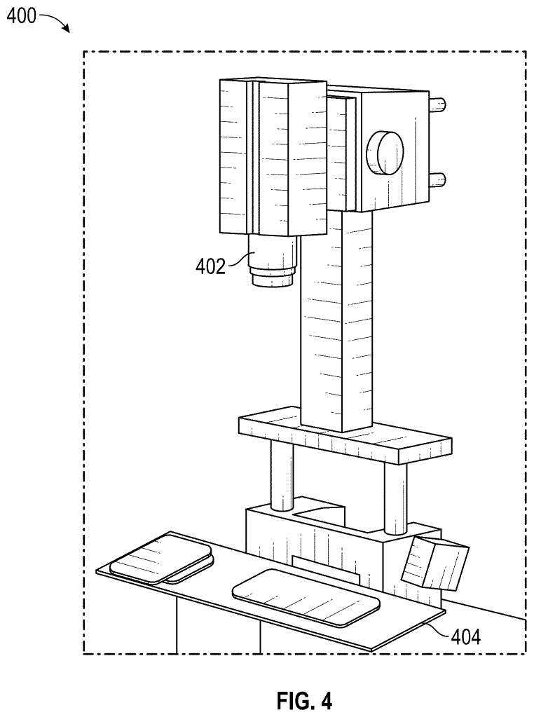

FIG. 5 depicts a schematic diagram of an image analysis module 500, including multiple layers of a neural network in accordance with aspects of the present disclosure. The image analysis module 500 may be or may be implemented by the image analysis system. The image analysis module can implement one or more machine learning algorithms in order to diagnose one or more diseases within image data provided as an input to the machine leaning algorithm 500 by identifying structures or features present in the image data that are consistent with training data used to train the machine learning algorithm. Further, the image analysis module 500 may correspond to one or more of a machine learning model, a convolutional neural network, etc. In the example of FIG. 4, the image analysis module 500 may correspond to a convolutional neural network.

The machine learning algorithm can include an input layer 502, one or more intermediate layer(s) 504 (also referred to as hidden layer(s)), and an output layer 506. The input layer 502 may be an array of pixel values. For example, the input layer may include a 320×320×3 array of pixel values. Each value of the input layer 502 may correspond to a particular pixel value. Further, the input layer 502 may obtain the pixel values corresponding to the image. Each input of the input layer 502 may be transformed according to one or more calculations.

Further, the values of the input layer 502 may be provided to an intermediate layer 504 of the machine learning algorithm. In some embodiments, the machine learning algorithm may include one or more intermediate layers 504. The intermediate layer 504 can include a plurality of activation nodes that each perform a corresponding function. Further, each of the intermediate layer(s) 504 can perform one or more additional operations on the values of the input layer 502 or the output of a previous one of the intermediate layer(s) 504. For example, the input layer 502 is scaled by one or more weights 503a, 503b, . . . , 503m prior to being provided to a first one of the one or more intermediate layers 504. Each of the intermediate layers 504 includes a plurality of activation nodes 504a, 504b, . . . , 504n. While many of the activation nodes 504a, 504b, . . . are configured to receive input from the input layer 502 or a prior intermediate layer, the intermediate layer 504 may also include one or more activation nodes 504n that do not receive input. Such activation nodes 504n may be generally referred to as bias activation nodes. When an intermediate layer 504 includes one or more bias activation nodes 504n, the number m of weights applied to the inputs of the intermediate layer 504 may not be equal to the number of activation nodes n of the intermediate layer 504. Alternatively, when an intermediate layer 504 does not includes any bias activation nodes 504n, the number m of weights applied to the inputs of the intermediate layer 504 may be equal to the number of activation nodes n of the intermediate layer 504.

By performing the one or more operations, a particular intermediate layer 504 may be configured to produce a particular output. For example, a particular intermediate layer 504 may be configured to identify an edge of a tissue sample and/or a block sample. Further, a particular intermediate layer 504 may be configured to identify an edge of a tissue sample and/or a block sample and another intermediate layer 504 may be configured to identify another feature of the tissue sample and/or a block sample. Therefore, the use of multiple intermediate layers can enable the identification of multiple features of the tissue sample and/or the block sample. By identifying the multiple features, the machine learning algorithm can provide a more accurate identification of a particular image. Further, the combination of the multiple intermediate layers can enable the machine learning algorithm to better diagnose the presence of a disease. The output of the last intermediate layer 504 may b e received as input at the output layer 506 after being scaled by weights 505a, 505b, 505m. Although only one output node is illustrated as part of the output layer 506, in other implementations, the output layer 506 may include a plurality of output nodes.

The outputs of the one or more intermediate layers 504 may be provided to an output layer 506 in order to identify (e.g., predict) whether the image data is indicative of a disease present in the tissue sample. In some embodiments, the machine learning algorithm may include a convolution layer and one or more non-linear layers. The convolution layer may be located prior to the non-linear layer(s).

In order to diagnose the tissue sample associated with image data, the image analysis module 500 may be trained to identify a disease. By such training, the trained image analysis module 500 is trained to recognize differences in images and/or similarities in images. Advantageously, the trained image analysis module 500 is able to produce an indication of a likelihood that particular sets of image data are indicative of a disease present in the tissue sample.

Training data associated with tissue sample(s) may be provided to or otherwise accessed by the image analysis module 500 for training. The training data may include image data corresponding to a tissue sample tissue block data that has previously been identified as having a disease. The image analysis module 500 trains using the training data set. The image analysis module 500 may be trained to identify a level of similarity between first image data and the training data. The image analysis module 500 may generate an output that includes a representation (e.g., an alphabetical, numerical, alphanumerical, or symbolical representation) of whether a disease present in a tissue sample corresponding to the first image data.

In some embodiments, training the image analysis module 500 may include training a machine learning model, such as a neural network, to determine relationships between different image data. The resulting trained machine learning model may include a set of weights or other parameters, and different subsets of the weights may correspond to different input vectors. For example, the weights may be encoded representations of the pixels of the images. Further, the image analysis system can provide the trained image analysis module 500 for image processing. In some embodiments, the process may be repeated where a different image analysis module 500 is generated and trained for a different data domain, a different user, etc. For example, a separate image analysis module 500 may be trained for each data domain of a plurality of data domains within which the image analysis system is configured to operate.

Illustratively, the image analysis system may include and implement one or more imaging algorithms. For example, the one or more imaging algorithms may include one or more of an image differencing algorithm, a spatial analysis algorithm, a pattern recognition algorithm, a shape comparison algorithm, a color distribution algorithm, a blob detection algorithm, a template matching algorithm, a SURF feature extraction algorithm, an edge detection algorithm, a keypoint matching algorithm, a histogram comparison algorithm, or a semantic texton forest algorithm. The image differencing algorithm can identify one or more differences between first image data and second image data. The image differencing algorithm can identify differences between the first image data and the second image data by identifying differences between each pixel of each image. The spatial analysis algorithm can identify one or more topological or spatial differences between the first image data and the second image data. The spatial analysis algorithm can identify the topological or spatial differences by identifying differences in the spatial features associated with the first image data and the second image data. The pattern recognition algorithm can identify differences in patterns of the first image data and the training data. The pattern recognition algorithm can identify differences in patterns of the first image data and patterns of the training data. The shape comparison algorithm can analyze one or more shapes of the first image data and one or more shapes of the second image data and determine if the shapes match. The shape comparison algorithm can further identify differences in the shapes.

The color distribution algorithm may identify differences in the distribution of colors over the first image data and the second image data. The blob detection algorithm may identify regions in the first image data that differ in image properties (e.g., brightness, color) from a corresponding region in the training data. The template matching algorithm may identify the parts of first image data that match a template (e.g., training data). The SURF feature extraction algorithm may extract features from the first image data and the training data and compare the features. The features may be extracted based at least in part on particular significance of the features. The edge detection algorithm may identify the boundaries of objects within the first image data and the training data. The boundaries of the objects within the first image data may be compared with the boundaries of the objects within the training data. The keypoint matching algorithm may extract particular keypoints from the first image data and the training data and compare the keypoints to identify differences. The histogram comparision algorithm may identify differences in a color histogram associated with the first image data and a color histogram associated with the training data. The semantic texton forests algorithm may compare semantic representations of the first image data and the training data in order to identify differences. It will be understood that the image analysis system may implement more, less, or different imaging algorithms. Further, the image analysis system may implement any imaging algorithm in order to identify differences between the first image data and the training data.

### Normalizing the Image Data of a Tissue Sample Obtained Using Multispectral Imaging

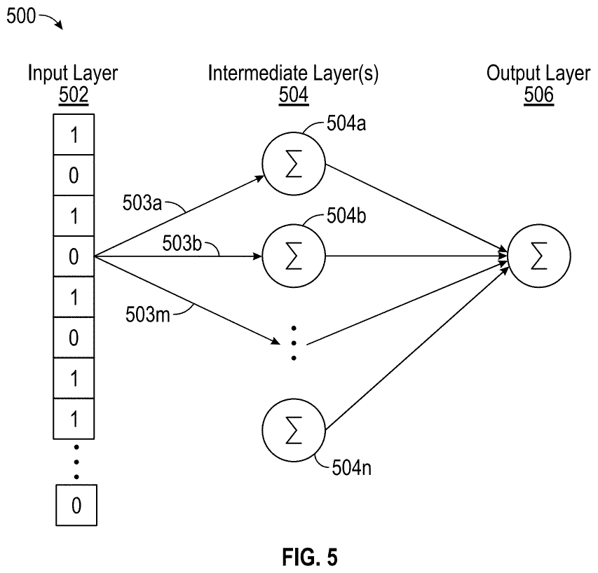

FIG. 6 is an example method 600 for normalizing image data of a tissue sample obtained using multispectral imaging in accordance with aspects of this disclosure. The method 600 may be performed by a multispectral imaging system, such as the system 100 of FIG. 1. Depending on the implementation, the blocks of the method 600 may be performed by different components of the system 100, such as the imaging device 102, the image analysis system 108, and/or the user computing device 110. For simplicity, aspects of the method 600 will be described simply as performed by the multispectral imaging system 100.

The method 600 begins at block 601. At block 602, the multispectral imaging system performs a multispectral scan of a tissue sample to generate image data. At block 604, the multispectral imaging system identifies wavelengths in the image data for which a threshold number of pixels have a gradient value in a same orientation vector. For example, the threshold number of pixels having the gradient in the same orientation vector may be indicative of the presence of a consistent gradient value in the image data due to processing of the tissue sample (e.g., due to fixation of the tissue sample using formalin or another chemical composition used for tissue sample fixation).

At block 606, the multispectral imaging system preprocess the image data to generate a normalization factor based on pixel values of the image data within at least one sub-region of the image data. In some implementations, the preprocessing of the image data to generate the normalization factor may be further based at least in part on wavelengths identified in block 604. In certain implementations, the normalization factor may be determined at each pixel location within the image data.

At block 608, the multispectral imaging system provides the preprocessed image data and the normalization factor as inputs to a machine learning algorithm. In some implementations, the normalization factor may be used to scale each pixel within the image data. That is, the normalization factor can be used to scale the spectrum at each pixel location within the image data.

At block 610, the multispectral imaging system may determine, based on an output of the machine learning algorithm, whether the image data is indicative of a disease present in the tissue sample. For example, the machine learning algorithm may be configured to detect the presence of one or more diseases based on processing the preprocessed input image data. The method ends at block 612.

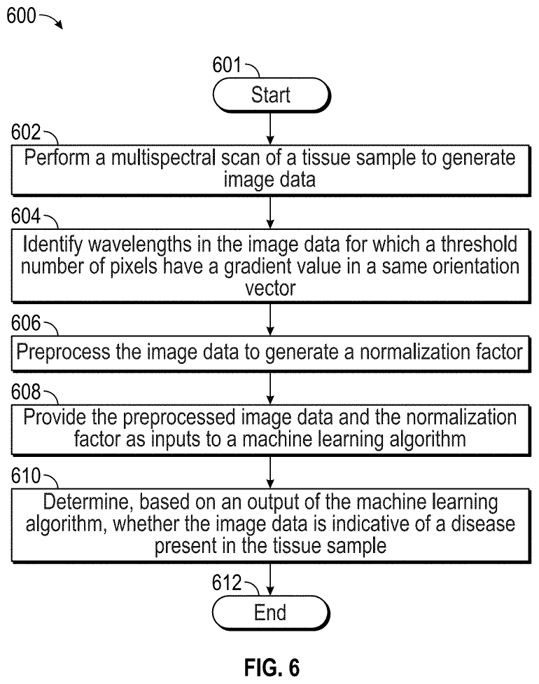

FIG. 7 is an example method 700 for preprocessing the image data as a part of the method of FIG. 6 in accordance with aspects of this disclosure. In particular, the method 700 is one example implementation of block 606 of method 700.

The method 700 begins at block 701. At block 702, the multispectral imaging system subtracts at least one mean of the pixel values within the at least one sub-region of the image data from the pixel values. For example, in certain implementations, the multispectral imaging system subtracting the mean of each of three color channels (e.g., RGB, YUV, CMYK, etc.) from the image data.

At block 704, the multispectral imaging system initializes a plurality of weights applied to one or more inputs of the activation nodes of the machine learning algorithm. The plurality of weights may be selected to facilitate confining the activation nodes within a defined Gaussian range.

At block 706, the multispectral imaging system takes a random sample of input data to a current one of the intermediate layer(s) of the machine learning algorithm. At block 708, the multispectral imaging system calculates a mean and a variance of the random sample of the input data. At block 710, the multispectral imaging system provide the mean and the variance as inputs to the a current one of the intermediate layers.

The method 700 may repeat blocks 706-710 for each intermediate layer beyond the first intermediate layer of the machine learning algorithm. Method 700 may have the effect of normalizing the inputs for each of the intermediate layers. The method 700 ends at block 712.

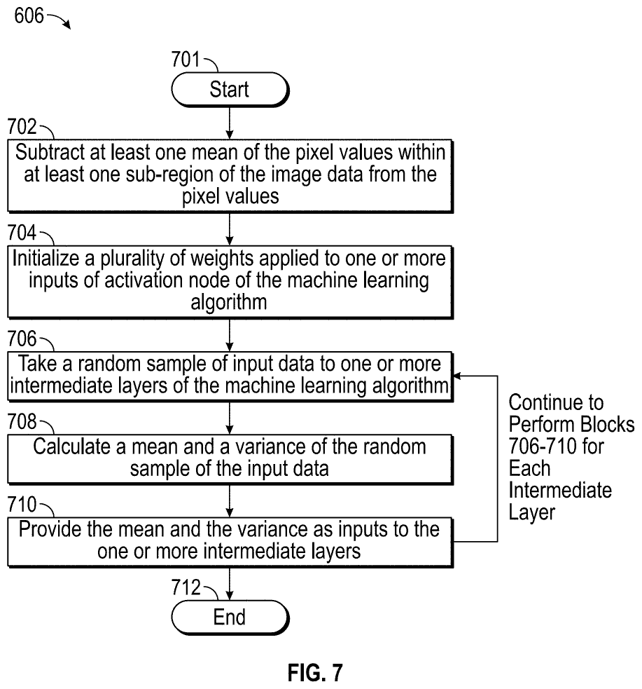

FIG. 8 is an example method 800 for scaling input data provided to activation nodes of a machine learning algorithm as a part of the method of FIG. 6 in accordance with aspects of this disclosure. In particular, the method 800 is one example implementation of block 608 of method 700.

The method 800 begins at block 801. At block 802, the multispectral imaging system scales input data to a first one of the one or more intermediate layers by the normalization factor determined in block 606. The first intermediate layer may be located after a convolution layer but before any of one or more non-linear layers. In certain implementations, the machine learning algorithm includes a plurality of feature dimensions and a plurality of spatial locations. In these implementations, the normalization factor can be applied individually for each of the feature dimensions and jointly for each of the spatial dimensions. This may result in an activation map representing the machine learning algorithm having one mean and one variance, which leads to normalization of the activation nodes in the same manner as nearby activation nodes.

At block 804, the multispectral imaging system uses the normalized feature dimensions and normalized spatial dimensions determined in block 802 to scale and shift inputs to each of the activation nodes of the machine learning algorithm. Using the method 800, the multispectral imaging system is able to scale the input data by the normalization factor. The method 800 ends at block 806.

Aspects of this disclosure address both the initialization as well as the normalization of one or more intermediate layers of the machine learning algorithm. For example, at initialization, the preprocessing of the image data to determine a normalization factor enables the system to preserve the structure of the tissue sample captured by the image data, which can improve the accuracy of the machine learning algorithm any parameters of the machine learning algorithm are determined.

For successive intermediate layers, aspects of this disclosure can compute gradients within the image data by scaling and shifting each activation node using the determined parameters. This enables the machine learning algorithm to control the degree of gradient saturation and preserve spatial structure. In particular, the combination of preprocessing and scaling the image data by the normalization factor after the convolution layer and before any non-linear layers provides the advantage of enabling the machine learning algorithm to operate with a limit to a regime of unit-gaussian input scaling into the activation nodes function used for initialization. Thus, aspects of this disclosure have greater fine-tuned control over saturation as the machine learning algorithm becomes deeper due to progressively multiplying by updated weights.

Those skilled in the art will recognize that this disclosure provide numerous other advantages over other machine learning algorithms, including higher learning rates, implicitly regularizing which reduces potential to overfit, improving gradient flow through the network, and/or reducing reliance on initialization which saturates gradients at each layer.

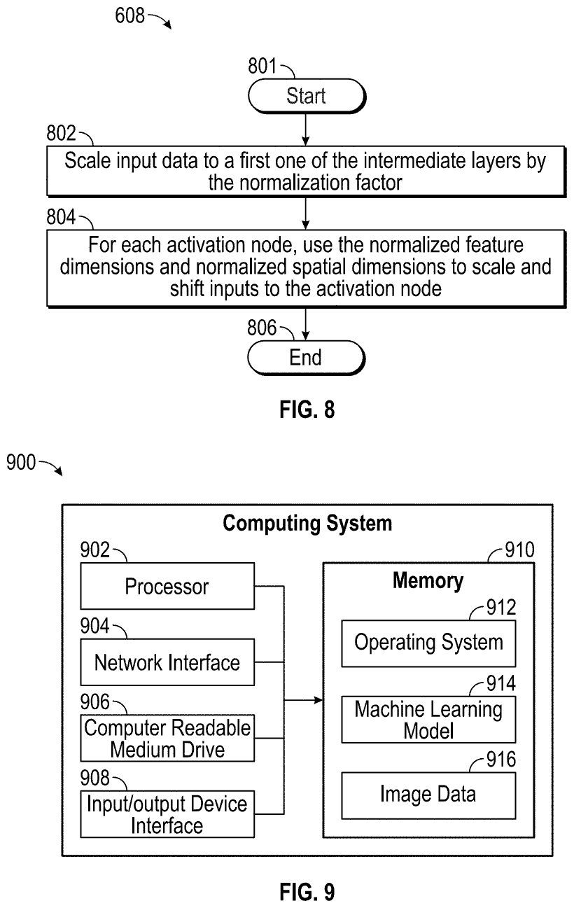

FIG. 9 is an example computing system 900 which can implement any one or more of the imaging device 102, image analysis system 108, and user computing device 110 of the multispectral imaging system illustrated in FIG. 1. The computing system 900 may include: one or more computer processors 902, such as physical central processing units (“CPUs”); one or more network interfaces 904, such as a network interface cards (“NICs”); one or more computer readable medium drives 906, such as a high density disk (“HDDs”), solid state drives (“SDDs”), flash drives, and/or other persistent non-transitory computer-readable media; an input/output device interface 908, such as an input/output (“IO”) interface in communication with one or more microphones; and one or more computer readable memories 910, such as random access memory (“RAM”) and/or other volatile non-transitory computer-readable media.

The network interface 904 can provide connectivity to one or more networks or computing systems. The computer processor 902 can receive information and instructions from other computing systems or services via the network interface 904. The network interface 904 can also store data directly to the computer-readable memory 910. The computer processor 902 can communicate to and from the computer-readable memory 910, execute instructions and process data in the computer readable memory 910, etc.

The computer readable memory 910 may include computer program instructions that the computer processor 902 executes in order to implement one or more embodiments. The computer readable memory 910 can store an operating system 912 that provides computer program instructions for use by the computer processor 902 in the general administration and operation of the computing system 900. The computer readable memory 910 can further include computer program instructions and other information for implementing aspects of the present disclosure. For example, in one embodiment, the computer readable memory 910 may include a machine learning model 914 (also referred to as a machine learning algorithm). As another example, the computer-readable memory 910 may include image data 916. In some embodiments, multiple computing systems 900 may communicate with each other via respective network interfaces 904, and can implement multiple sessions each session with a corresponding connection parameter (e.g., each computing system 900 may execute one or more separate instances of the method 600), in parallel (e.g., each computing system 900 may execute a portion of a single instance of the method 600), etc.

### CONCLUSION

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the disclosure should not be taken to imply that the terminology is being re-defined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures can be combined, interchanged or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

Directional terms used herein (e.g., top, bottom, side, up, down, inward, outward, etc.) are generally used with reference to the orientation shown in the figures and are not intended to be limiting. For example, the top surface described above can refer to a bottom surface or a side surface. Thus, features described on the top surface may be included on a bottom surface, a side surface, or any other surface.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

The above description discloses several methods and materials of the present invention(s). This invention(s) is susceptible to modifications in the methods and materials, as well as alterations in the fabrication methods and equipment. Such modifications will become apparent to those skilled in the art from a consideration of this disclosure or practice of the invention(s) disclosed herein. Consequently, it is not intended that this invention(s) be limited to the specific embodiments disclosed herein, but that it cover all modifications and alternatives coming within the true scope and spirit of the invention(s) as embodied in the attached claims.

1. An image analysis apparatus, comprising:
a memory coupled to an imaging device; and
a hardware processor configured to:
receive image data from the imaging device, the image data representative of a tissue sample,
process the image data to generate a normalization factor based on pixel values of the image data within at least one sub-region of the image data,
provide the preprocessed image data and the normalization factor as inputs to a machine learning algorithm, and
determine, based on an output of the machine learning algorithm, whether the image data is indicative of a disease present in the tissue sample.

2. The image analysis apparatus of claim 1, wherein the preprocessing the image data comprises:
subtracting at least one mathematical mean of the pixel values within the at least one sub-region of the image data from the pixel values.

3. The image analysis apparatus of claim 2, wherein the at least one mathematical mean comprises a mathematical mean for each of three color channels of pixels values within the at least one sub-region.

4. The image analysis apparatus of claim 1, wherein the hardware processor is further configured to:
initialize a plurality of weights applied to one or more inputs of activation nodes of the machine learning algorithm, wherein the plurality of weights facilitate confining the activation nodes within a defined Gaussian range.

5. The image analysis apparatus of claim 1, wherein:
the machine learning algorithm is configured to interface with a plurality of layers of a neural network including an initial layer, one or more intermediate layers, and an output layer, and
the hardware processor is further configured to, for each of the one or more intermediate layers:
take a random sample of input data to the one or more intermediate layers, calculate a mean and a variance of the random sample of the input data, and
provide the mean and the variance as inputs to the one or more intermediate layers.

6. The image analysis apparatus of claim 5, wherein:
the one or more intermediate layers comprise a convolution layer and one or more non-linear layers, and
the hardware processor is further configured to, for each of the one or more intermediate layers, scale input data to a corresponding intermediate layer by the normalization factor, the corresponding intermediate layer located after the convolution layer but before the one or more non-linear layers.

7. The image analysis apparatus of claim 6, wherein:
the machine learning algorithm comprises a plurality of feature dimensions and a plurality of spatial locations, and
the normalization factor is applied individually for each of the feature dimensions and jointly for each of the spatial dimensions.

8. The image analysis apparatus of claim 7, wherein:
each of the one or more intermediate layers comprises one or more activation nodes, and
the hardware processor is further configured to, for each activation node,
use the normalized feature dimensions and normalized spatial dimensions to scale and shift inputs to the activation node.

9. The image analysis apparatus of claim 1, wherein the hardware processor is further configured to:
identify wavelengths in the image data for which a threshold number of pixels have a gradient value in a same orientation vector.

10. The image analysis apparatus of claim 1, further comprising:
an imaging device configured to perform a multispectral scan of the tissue sample to generate the image data.

11. (canceled)

12. A non-transitory computer-readable medium having stored thereon instructions which, when executed by a hardware processor, cause the hardware processor to:
perform a multispectral scan of a tissue sample to generate image data;
preprocess the image data to generate a normalization factor based on pixel values of the image data within at least one sub-region of the image data;
provide the preprocessed image data and the normalization factor as inputs to a machine learning algorithm; and
determine, based on an output of the machine learning algorithm, whether the image data is indicative of a disease present in the tissue sample.

13. The non-transitory computer-readable medium of claim 2, wherein the preprocessing the image data comprises:
subtracting at least one mathematical mean of the pixel values within the at least one sub-region of the image data from the pixel values.

14. The non-transitory computer-readable medium of claim 13, wherein the at least one mathematical mean comprises a mathematical mean for each of three color channels of pixels values within the at least one sub-region.

15. The non-transitory computer-readable medium of claim 2, wherein the instructions are further configured to cause the hardware processor to:
initialize a plurality of weights applied to one or more inputs of activation nodes of the machine learning algorithm, wherein the plurality of weights facilitate confining the activation nodes within a defined Gaussian range.

16. The non-transitory computer-readable medium of claim 2, wherein:
the one or more intermediate layers comprise a convolution layer and one or more non-linear layers, and
the method further comprises, for each of the one or more intermediate layers, scale input data to a corresponding intermediate layer by the normalization factor, the corresponding intermediate layer located after the convolution layer but before the one or more non-linear layers.

17. A method of determining whether a disease is present in a tissue sample, comprising:
performing a multispectral scan of the tissue sample to generate image data;
preprocessing the image data to generate a normalization factor based on pixel values of the image data within at least one sub-region of the image data;
providing the preprocessed image data and the normalization factor as inputs to a machine learning algorithm; and
determining, based on an output of the machine learning algorithm, whether the image data is indicative of a disease present in the tissue sample.

18. The method of claim 17, wherein the preprocessing the image data comprises:
subtracting at least one mathematical mean of the pixel values within the at least one sub-region of the image data from the pixel values.

19. The method of claim 18, wherein the at least one mathematical mean comprises a mathematical mean for each of three color channels of pixels values within the at least one sub-region.

20. The method of claim 17, further comprising:
initializing a plurality of weights applied to one or more inputs of activation nodes of the machine learning algorithm, wherein the plurality of weights facilitate confining the activation nodes within a defined Gaussian range.
